(19) United States
(12) Patent Application Publication
(10) Pub. No.: US 2022/0226546 A1
(43) Pub. Date: Jul. 21, 2022

(54) SURFACE COATING STRUCTURE OF SURGICAL PROSTHESIS AND METHOD FOR MODIFYING SURFACE OF SURGICAL PROSTHESIS USING SAME

(71) Applicant: OID LIMITED, Seoul (KR)

(72) Inventors: Jung Mok Seo, Seoul (KR); Woo Young Jang, Seoul (KR)

(21) Appl. No.: 17/616,592

(22) PCT Filed: Jun. 4, 2020
(86) PCT No.: PCT/KR2020/007242

§ 371 Date: Dec. 3, 2021

(30) Foreign Application Priority Data
Jun. 5, 2019 (KR) 10-2019-0066594
Jul. 3, 2019 (KR) 10-2019-0079920

(51) Int. Cl. A61L 31/08; A61B 17/72; A61L 31/16

(52) U.S. Cl. CPC A61L 31/08; A61B 17/72; A61L 31/16; A61L 2420/08; A61B 2017/00938; A61L 2400/12; A61L 2400/10; A61L 2300/424; A61L 2430/02; A61L 2420/02

(57) ABSTRACT

A surface coating structure of a surgical prosthesis according to an exemplary embodiment of the present disclosure may include: a first coating layer formed on the surface of the surgical prosthesis and including an amino compound for surface adhesion; a second coating layer formed on one side of the first coating layer and including a fluorine compound conferring hydrophobicity to the surface coating structure of the surgical prosthesis; and a third coating layer formed on one side of the second coating layer and including a lubricant component for preventing adhesion of a biomaterial existing in a subject into which the surgical prosthesis is inserted.

### TECHNICAL FIELD

The present disclosure relates to a surface coating structure of a surgical prosthesis, a surface-modified surgical prosthesis and a method for modifying the surface of a surgical prosthesis based thereon.

### BACKGROUND ART

Post-orthopedic surgery infection is one of important complications that may prolong treatment period and even cause death. It may also cause extended hospitalization and associated legal issues. The cause of infections occurring after orthopedic surgery includes the health condition of a patient such as diabetes, immunodeficiency, malnutrition, etc., environmental factors during the surgery, and the like.

In particular, prostheses are used frequently in orthopedic surgeries. Infection around the prosthesis, although infrequent, is not easy to treat and the prosthesis has to be removed for treatment in most cases. In such infection, it is a critical issue whether microorganisms are attached to the surface of the prosthesis.

When microorganisms are attached to the surface of the prosthesis, a biofilm may be formed by a combination of the proliferating microorganisms and the substances secreted by the microorganisms. Although antibiotics, etc. are used to eliminate the microorganisms present inside the biofilm, a large amount of the antibiotics, etc. enough to eliminate the microorganisms fail to reach the microorganisms. Therefore, methods for preparing prostheses in consideration of this difficulty are being developed.

For example, Korean Patent Publication No. 10-2008-0068853, published on Jul. 24, 2008, discloses a method of depositing discrete nanoparticles on the surface of an implant.

### DISCLOSURE OF THE INVENTION

### Technical Problem

The present disclosure is directed to providing a surface coating structure of a surgical prosthesis, a surface-modified surgical prosthesis and a method for modifying the surface of a surgical prosthesis based thereon.

### Technical Solution

A surface coating structure of a surgical prosthesis according to an exemplary embodiment of the present disclosure may include a first coating layer formed on the surface of the surgical prosthesis and including an amino compound for surface adhesion, a second coating layer formed on one side of the first coating layer and including a fluorine compound conferring hydrophobicity to the surface coating structure of the surgical prosthesis and a third coating layer formed on one side of the second coating layer and including a lubricant component for preventing adhesion of a biomaterial existing in a subject into which the surgical prosthesis is inserted.

A surface-modified prosthesis according to another exemplary embodiment of the present disclosure may include a prosthesis inserted into a fracture site to fix the fracture site, a first coating layer formed on the surface of the prosthesis and including an amino compound for surface adhesion, a second coating layer formed on one side of the first coating layer and including a fluorine compound conferring hydrophobicity to the surface coating structure of the surgical prosthesis and a third coating layer formed on one side of the second coating layer and including a lubricant component for preventing adhesion of a biomaterial existing in a subject into which the surgical prosthesis is inserted.

A method for modifying the surface of a surgical prosthesis according to another exemplary embodiment of the present disclosure may include a step of forming a first coating layer including an amino compound for surface adhesion on the surface of a surgical prosthesis for producing a surface coating structure of the surgical prosthesis, a step of forming a second coating layer including a fluorine compound conferring hydrophobicity to the surface coating structure of the surgical prosthesis on one side of the first coating layer and a step of forming a third coating layer including a lubricant component for preventing adhesion of a biomaterial existing in a subject into which the surgical prosthesis is inserted on one side of the second coating layer.

### Advantageous Effects

A surface coating structure of a surgical prosthesis, a surface-modified surgical prosthesis and a method for modifying the surface of a surgical prosthesis based thereon according to exemplary embodiments of the present disclosure can reduce the pain of a patient by not only preventing infection caused by attachment of bacteria on a prosthesis but also fundamentally preventing immune rejection by preventing the attachment of inflammatory factors such as blood proteins.

### BRIEF DESCRIPTION OF THE DRAWINGS

FIG. 1 shows reference images for describing the problem of an existing surgical prosthesis.

FIG. 2 schematically shows a surface-modified surgical prosthesis according to an exemplary embodiment of the present disclosure inserted into a subject as well as an enlarged view of a portion of the surface of the surgical prosthesis.

FIG. 3 is an enlarged view of a portion of the surface of the surgical prosthesis of FIG. 2.

FIG. 4 schematically shows a procedure of producing a surface coating structure of a surgical prosthesis according to an exemplary embodiment of the present disclosure.

FIG. 5 shows reference images illustrating the insertion of a prosthesis 10 into a fracture site of an animal according to an exemplary embodiment of the present disclosure.

FIG. 6 is a flowchart illustrating a method for modifying the surface of a surgical prosthesis according to an exemplary embodiment of the present disclosure.

FIG. 7 is a flowchart illustrating a method for modifying the surface of a surgical prosthesis according to another exemplary embodiment of the present disclosure.

FIG. 8 shows a result of testing the performance of an orthopedic prosthesis according to an exemplary embodiment of the present disclosure.

FIG. 9 shows a result of testing the performance of an orthopedic prosthesis according to an exemplary embodiment of the present disclosure.

### BEST MODE FOR CARRYING OUT THE INVENTION

The attached drawings and the following description are provided for those having ordinary knowledge in the art to fully understand the advantages of the present disclosure and the purpose achieved by carrying out the present disclosure.

Hereinafter, the specific exemplary embodiments of the present disclosure will be described in detail referring to the attached drawings. However, the present disclosure may be embodied in various different forms and is not limited by the described exemplary embodiments. In the drawings, the portions irrelevant of the description of the present disclosure will be omitted and like numerals indicate like elements.

Hereinafter, exemplary embodiments of the present disclosure are described in detail referring to the attached drawings. When describing the present disclosure, the detailed description of known related functions and components may be omitted to avoid unnecessarily obscuring the subject matter of the present disclosure.

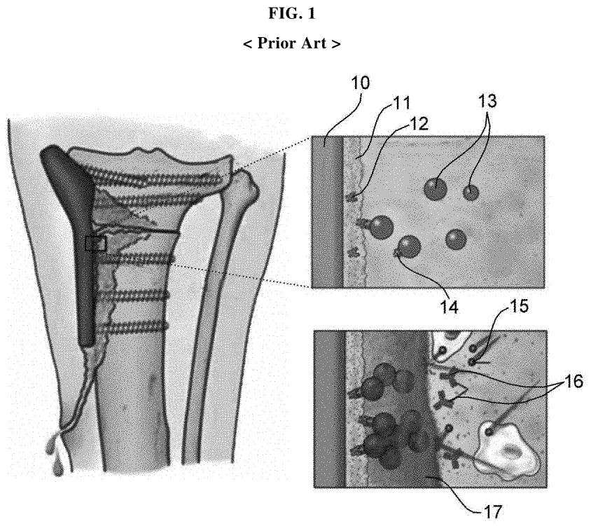

FIG. 1 shows reference images for describing the problem of an existing surgical prosthesis. As shown in FIG. 1, a surgical prosthesis 10 coated according to an existing method has the problem that, when a predetermined time has passed after insertion into a fracture site of the body, fibronectin molecules 12 are produced and a biofilm 17 is formed from the binding of the fibronectin molecules to receptors 14 bound to bacteria 13 in the body, and the biofilm 17 is not removed easily only with antibodies 16, antibiotics 15, etc.

Hereinafter, a surface coating structure of a surgical prosthesis and a method for modifying the surface of a surgical prosthesis according to an exemplary embodiment of the present disclosure are described in detail referring to the relevant drawings.

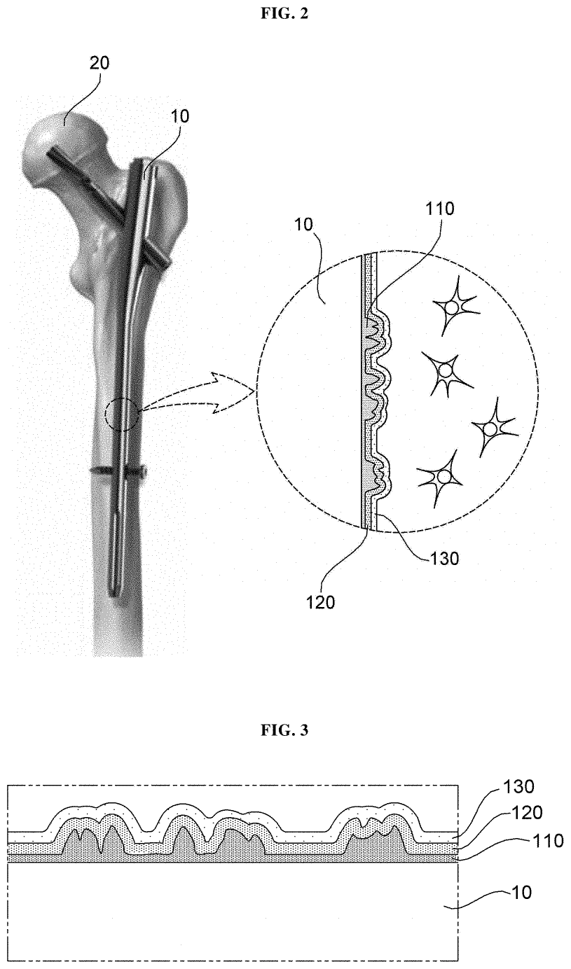

FIG. 2 schematically shows a surface-modified surgical prosthesis according to an exemplary embodiment of the present disclosure inserted into a subject as well as an enlarged view of a portion of the surface of the surgical prosthesis, and FIG. 3 is an enlarged view of a portion of the surface of the surgical prosthesis of FIG. 2.

Referring to FIG. 2, a surgical prosthesis 10 according to an exemplary embodiment of the present disclosure may consist of a first fixing peg 110, a second fixing peg 120 and a third fixing peg 130. For example, the subject may be human, an animal, etc., the first fixing peg 110 may be inserted into the femur, the second fixing peg 120 may be inserted into the acetabulum, and the third fixing peg may be inserted into a region below the femur.

The shape of the prosthesis is not limited to that shown in FIG. 2, and the surgical prosthesis 10 may have any shape corresponding to the target site of the subject. That is to say, the surgical prosthesis 10 may be used in various sites of the subject, including hip joint, elbow joint, knee joint, etc.

The surface of the prosthesis 10 of the present disclosure may be formed of at least one metal material. For example, the surface of the prosthesis may be formed of a metal material such as titanium (Ti), stainless steel, etc.

The surgical prosthesis 10 of the present disclosure is a metal material inserted into the fractured bone of the body. In order to fundamentally prevent the attachment (biofouling) of biomaterials causing side effects such as proteins, inflammatory factors, bacteria, etc. on the metal material, the surface of the prosthesis 10 is coated sequentially with three coating layers, i.e., a first coating layer 110, a second coating layer 120 and a third coating layer 130, as shown in FIG. 2 in order to reduce the pain of a patient by not only preventing infection caused by attachment of bacteria on the prosthesis 10 but also fundamentally preventing immune rejection by preventing the attachment of inflammatory factors such as blood proteins.

Referring to FIG. 2 and FIG. 3, a surface coating structure of the surgical prosthesis may include the first coating layer 110, the second coating layer 120 and the third coating layer 130.

Before coating the first to third coating layers on the surface of the surgical prosthesis 10, a process of pretreating the surgical prosthesis 10 and forming surface roughness on the surface of the pretreated prosthesis may be performed first, and then the first to third coating layers may be coated on the surface of the surgical prosthesis with the surface roughness formed.

The pretreatment process is a process wherein organic or inorganic materials present on the surface of the prosthesis are removed prior to the coating of the surface of the prosthesis, and the process of forming the surface roughness is a process for forming a space wherein a lubricant fluid of the third coating layer to be coated later can be retained physically, so that the prosthesis can operate for a long period of time after being inserted into the body of the subject.

In an exemplary embodiment of the present disclosure, the first coating layer 110 may be formed on the surface of the surgical prosthesis 10 and may include an amino compound for surface adhesion. That is to say, the first coating layer 110 may be a layer for forming amino groups, which are surface adhesion functional groups having strong binding ability to the second coating layer, on the surface of the prosthesis. The amino compound included in the first coating layer 110 may be at least one of 3-aminopropyltrimethoxysilane, 3-aminopropylethoxydimethylsilane, 3-aminopropyldiethoxymethylsilane, 3-[2-(2-aminoetylanmino)ethylamino]propyl-trimethoxysilane and polydopamine.

In an exemplary embodiment of the present disclosure, the amino compound included in the first coating layer 110 may include polydopamine. In another exemplary embodiment of the present disclosure, the first coating layer 110 may include an aminosilane compound. Here, the aminosilane compound may be at least one of 3-aminopropyltrimethoxysilane, 3-aminopropyltrimethoxysilane (APTMS) and 3-aminopropylethoxydimethylsilane.

First, the first coating layer 110 using an adhesion material including polydopamine will be described. The first coating layer 110 using polydopamine may consist of a first component, a second component and a third component. For example, the first component may be dopamine hydrochloride, the second component may be copper sulfate and the third component may be hydrogen peroxide. The first coating layer using polydopamine may be coated on the surface of the prosthesis 10 using a mixture solution of the first to third components.

In another exemplary embodiment of the present disclosure, the first coating layer 110 may be formed by formed by dipping in a mixture solution wherein a fourth component has been further added to the mixture solution of the first to third components. The fourth component may be a Tris buffer which serves as a solvent that dissolves the first to third components. The concentration of the solvent may be 40-60 mM, and the pH may be 8-9. Specifically, the concentration of the solvent may be 50 mM, and the pH may be 8.5.

The dopamine hydrochloride constituting the first coating layer 110 may form a nano- or microstructure on the surface of the surgical prosthesis through polymerization and can make the binding to the second coating layer 120 coated on the first coating layer 110 stronger through strong adhesion. The hydroxyl (—OH) and amino (—NH₂) functional groups contained in the polydopamine form chemical bonding with a fluorocarbon-based polymer of the second coating layer 120.

The polydopamine-based first coating layer 110, which serves as an adhesion layer, may be coated on the surface of the prosthesis 10 through a solution-based process, and a uniform coating of complicated shapes dot and mesh patterns and various materials may be formed on the surface of the orthopedic prosthesis.

In an exemplary embodiment of the present disclosure, the first component constituting the polydopamine-based first coating layer 110 may have a chemical formula of (HO)₂C₆H₃CH₂CH₂NH₂—HCl and may have a molecular weight of 189.64 g/mol.

The second component may be copper sulfate (CuSO₄.5H₂O) and may reduce processing time by facilitating the polymerization of polydopamine on the surface of the prosthesis through oxidation.

The third component may be hydrogen peroxide (H₂O₂), which produces reactive oxygen species such as O₂⁻, HO₂⁻ and OH⁻ by reacting with Cu²⁺ of copper sulfate. The produced reactive oxygen species facilitate the polymerization of polydopamine and improve deposition rate greatly.

In an exemplary embodiment, the first coating layer 110 using polydopamine may specifically have a thickness of 30-50 nm. The coating thickness increases with the dipping time of the mixture solution. If the coating is performed for a short period of time, the first coating layer including polydopamine may not be deposited due to insufficient polymerization. And, if the dipping is performed for too long a period of time, the nano- or microstructure formed on the surface of the prosthesis becomes smooth and the coating may be peeled easily.

In another exemplary embodiment of the present disclosure, the first coating layer 110 may also include 3-aminopropyltrimethoxysilane (hereinafter, APTES), which is an aminosilane compound. Hereinafter, the first coating layer 110 using an adhesion material including APTES will be described.

In this exemplary embodiment, the first coating layer 110 may be coated as follows. Before dipping in a mixture solution of APTES, the surgical prosthesis may be treated first with oxygen plasma to form hydroxyl (—OH) groups as intermediate bridges necessary for the formation of the structure of APTES.

Then, the first coating layer may be coated by dipping the surface of the prosthesis with hydroxyl groups formed through oxygen plasma treatment in a mixture solution of APTES and ethanol.

The first coating layer 110 using APTES may consist of a first component and a second component. For example, the first component may be one of N-β-(aminoethyl)-7-aminopropyltrimethoxysilane, 1,3,5-tris[2-(trimethoxysilyl)propyl]-1,3,5-triazine-2,4,6(1H,3H,5H)-trione (TTMSPI), and APTES (3-aminopropyltriethoxysilane), which are aminosilane compounds. Specifically, the first component may be APTES (3-aminopropyltriethoxysilane) and the second component may be ethanol. The first coating layer using APTES may be coated on the surface of the prosthesis 10 using a mixture solution of the first component and the second component. The first coating layer 110 including APTES may be coated on the surface of the prosthesis by dipping in a mixture solution of 2-10% of the first component and 90-98% of the second component. Most specifically, the first coating layer 110 may be formed by dipping the surface of the prosthesis in a mixture solution of 5% of the first component and 95% of the second component.

The APTES-based first coating layer 110 also serves as a coating layer which makes the binding to the second coating layer 120 stronger through strong adhesion, like the polydopamine-based coating layer. The —NH₂ functional groups contained in the APTES form chemical bonding with an amorphous fluoropolymer of the second coating layer.

In an exemplary embodiment of the present disclosure, the first component constituting the APTES-based first coating layer 110 may have a chemical formula of H₂N(CH₂)₃SI(OC₂H₅)₃ and may have a molecular weight of 221.37 g/mol.

The second component may be ethanol and may make the APTES to be polymerized uniformly on the surface of the prosthesis.

The first coating layer 110 using APTES may specifically have a thickness of 8-80 nm. The coating thickness increases with the dipping time of the mixture solution of the first and second components. If the coating is performed for a short period of time, the first coating layer 110 including APTES may not be deposited sufficiently due to insufficient polymerization.

The first coating layer may also be formed by treating with self-assembled monolayers having terminal —NH₂ functional groups. The self-assembled monolayer having terminal —NH₂ functional groups may be selected from a group consisting of 3-aminopropyltrimethoxysilane, 3-aminopropylethoxydimethylsilane, 3-aminopropyldiethoxymethylsilane and 3-[2-(2-aminoetylanmino)ethylamino]propyl-trimethoxysilane.

In an exemplary embodiment of the present disclosure, the second coating layer 120 may be formed on one side of the first coating layer 110 and may include a fluorine compound conferring hydrophobicity to the coating structure coating the surface of the surgical prosthesis 10.

If the second coating layer 120 is formed between the first coating layer 110 and the third coating layer 130, it can prevent the damage of the surface of the surgical prosthesis 10 caused by external impact or abrasion and can recover the distribution balance of the third coating layer 130 on the first coating layer 110 through spontaneous formation of the second coating layer 120 even when the distribution balance of the third coating layer 130 on the first coating layer 110 is broken.

Hereinafter, the second coating layer 120 using an adhesion material including the fluorine compound is described. The second coating layer 120 including the fluorine compound may consist of a first component and a second component. For example, the first component may be a polymer consisting of fluorine and carbon, and the second component may be selected from a group consisting of a perfluoroalkane, a perfluorodialkyl ether and a perfluorotrialkylamine. The second coating layer using the fluorine compound may be coated on the first coating layer 110 by dipping the prosthesis 10 coated with the first coating layer 110 in a mixture solution of the first component and the second component.

The fluorocarbon polymer constituting the second coating layer 120 serves to make the surface of the surgical prosthesis hydrophobic and maintain the third coating layer for a long time due to chemical affinity of the second coating layer 120 to the third coating layer 130.

In an exemplary embodiment of the present disclosure, the first component constituting the polydopamine-based second coating layer 120 may have a chemical formula of [C₆F₁₀O]_{N} and may have a molecular weight of 278.05 g/mol.

In an exemplary embodiment of the present disclosure, the first component constituting second coating layer 120 may be selected from fluorocarbons containing carboxyl (—COOH) functional groups for forming chemical bonding with the hydroxyl (—OH) and amino group (—NH2) functional groups formed on the first coating layer 110. For example, the first component constituting second coating layer 120 of the present disclosure may be one of perfluorodecanoic acid, perfluorooctanoic acid, trifluoroacetic acid and perfluorocarboxylic acid.

In an exemplary embodiment, the second coating layer 120 including the fluorine compound may have a thickness of specifically 100-200 nm.

In this exemplary embodiment, the second component may be selected from a group (fluorine-based solvent) consisting of a perfluoroalkane, a perfluorodialkyl ether and a perfluorotrialkylamine. The second component is mixed with the first component and controls the concentration of the mixture solution. As the content of the second component in the mixture solution is higher, the coating thickness of the second coating layer is decreased.

In an exemplary embodiment of the present disclosure, the third coating layer 130 may be formed on the second coating layer 120 and may include a lubricant component for reducing abrasion of the surgical prosthesis 10.

The third coating layer 130 is a lubricating layer serving as a lubricant and may wet the surface of the surface coating structure of the surgical prosthesis 10. As a result, microorganisms such as bacteria, etc. may slip on the surface of the prosthesis 10 without being attached to the surface of the surgical prosthesis 10.

The third coating layer 130 may be coated on the second coating layer 120 to have a predetermined surface energy. The lubricant fluid constituting the third coating layer 130 may have a low surface energy suitable to modify the surface of the surgical prosthesis 10. For example, the lubricant fluid may be a liquid perfluorocarbon.

In another exemplary embodiment, the lubricant fluid may be one of perfluorotri-n-pentylamine such as FC-70, perfluoropolyether such as Krytox-100 or Krytox-103, perfluorodecalin such as Flutec PP6, Fluorinert™ FC-70 or FC-40, perfluorohexane such as FC-72, perfluorooctane such as PF5080, perfluorooctyl bromide such as 1-bromoperfluorooctane, perfluoroperhydrophenanthrene such as Vitreon or FluoroMed APF-215HP, 3-ethoxy-1,1,1,2,3,4,4,5,5,6,6,6-dodecafluoro-2-trifluoromethylhexane such as HFE-7500, Krytox FG-40, Krytox-105 or Krytox-107 and perfluorodecalin.

For example, the lubricant fluid may have a viscosity of 0.1-0.8 cm²/s and a density of 1500-2000 kg/m³. Considering that the prosthesis 10 is inserted into a subject, the lubricant fluid constituting the third coating layer 130, which has the viscosity and density characteristics described above, can improve the repellency of the third coating layer 130 against microorganisms and improve the slipping of microorganisms on the third coating layer 130.

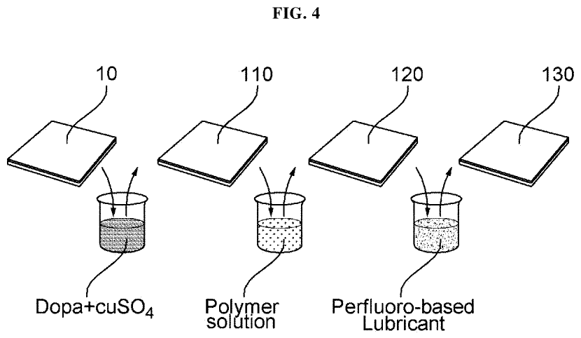

FIG. 4 schematically shows a procedure of producing a surface coating structure of a surgical prosthesis according to an exemplary embodiment of the present disclosure. First, referring to FIG. 4, a first coating layer 110 may be coated on a substrate 10 on which the surface of the prosthesis will be formed by dipping in a mixture solution of dopamine hydrochloride and copper sulfate. Then, a second coating layer 120 may be coated on the first coating layer 110 by dipping the surface of the prosthesis with the first coating layer coated in a mixture solution of a fluorocarbon polymer. Then, a third coating layer 130 may be coated on the second coating layer 120 by dipping the surface of the prosthesis with the second coating layer coated in a perfluorocarbon-based lubricant fluid.

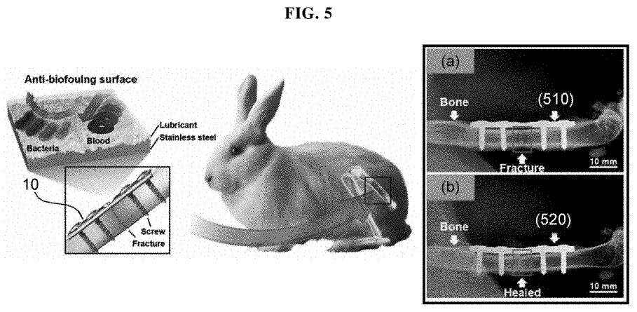

FIG. 5 shows reference images illustrating the insertion of the prosthesis 10 into a fracture site of an animal according to an exemplary embodiment of the present disclosure. In FIG. 5, (a) shows a prosthesis 510 with no coating layer inserted into a fracture site, and (b) shows a prosthesis 520 coated with a coating structure of the present inserted into a fracture site.

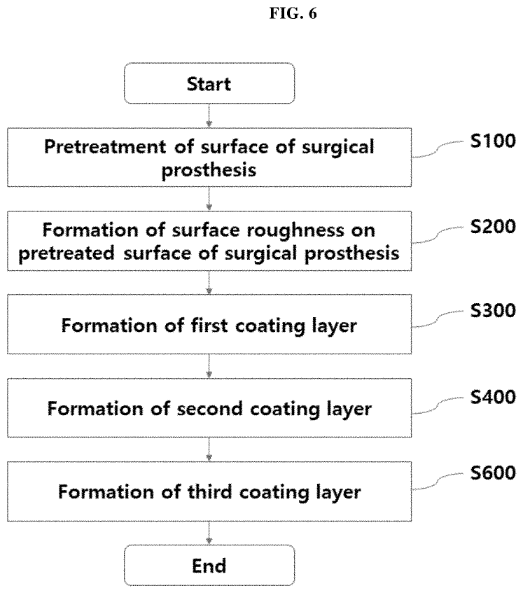

FIG. 6 is a flowchart illustrating a method for modifying the surface of a surgical prosthesis according to an exemplary embodiment of the present disclosure.

Referring to FIG. 6, in a method for modifying the surface of a prosthesis according to an exemplary embodiment of the present disclosure, the surface of a surgical prosthesis is pretreated first (S100). The pretreatment process is a process for removing organic or inorganic materials present on the surface of the prosthesis and washing the same. The prosthesis is dipped in acetone, which is an organic solvent, to remove organic materials present on the surface of the prosthesis, and the prosthesis is dipped in alcohol, which is an organic solvent, to remove organic materials once again. Then, the prosthesis is dipped in deionized water, which is an inorganic solvent, to remove the acetone and the alcohol that have been used to remove the organic materials on the surface of the prosthesis and to remove polar inorganic materials at the same time.

Then, surface roughness is formed on the pretreated surface of the surgical prosthesis (S200). In an exemplary embodiment, the surface roughness may be formed on the pretreated surface of the prosthesis by spraying polygonal crushed grits onto the pretreated surface of the surgical prosthesis together with compressed air.

For example, the crushed grits may have a size of specifically 2.0-3.3 μm (microns). The surface roughness formed on the surface of the prosthesis increases with the size of the crushed grits. But, if the size of the crushed grits is larger than 3.3 μm, the surface of the prosthesis may be damaged. In contrast, if the size of the crushed grits is smaller than 2.0 μm, it is not easy to form surface roughness on the surface of the prosthesis and, thus, a space wherein the lubricant fluid of the third coating layer can be retained.

The crushed grits may be sprayed for 120-300 seconds. The surface roughness formed on the surface of the prosthesis may increase with the spraying time. Of course, the spraying time of the crushed grits may be changed adequately within 120-300 seconds depending on the material or strength of the surface of the prosthesis.

Next, a first coating layer is formed on the surface of the surgical prosthesis with the surface roughness formed (S300). As described above referring to FIG. 2 and FIG. 3, the first coating layer may be formed on the surface of the prosthesis of the present disclosure by two methods.

As a first method, the first coating layer 110 may be coated based on an adhesion material including polydopamine. According to the first method, the first coating layer 110 may be coated by dipping the surface of the prosthesis with the surface roughness formed in a mixture solution of dopamine hydrochloride, copper sulfate and hydrogen peroxide in a Tris buffer. A detailed description will be omitted to avoid redundancy because it was given above with reference to FIG. 2 and FIG. 3.

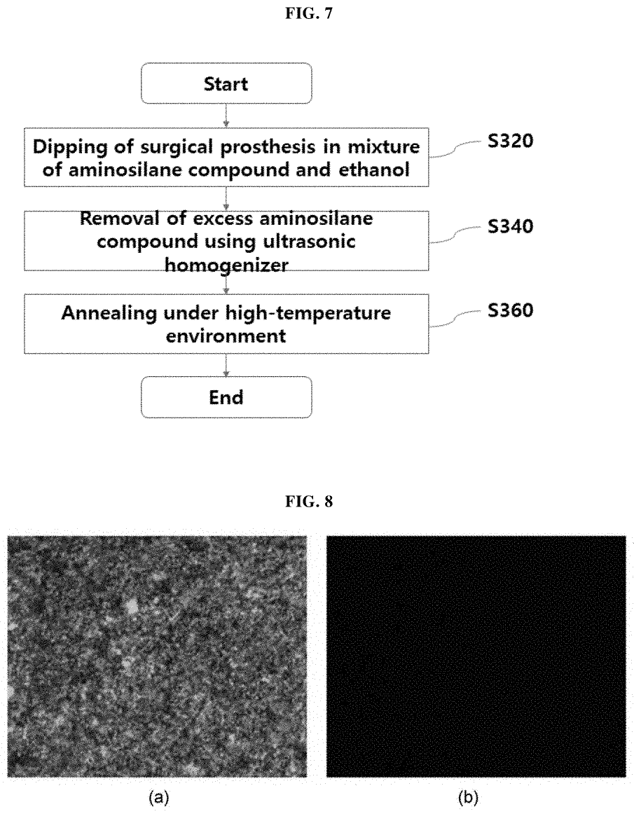

As a second method, the first coating layer 110 may be coated based on an adhesion material including APTES. A coating process of the first coating layer using APTES will be described in more detail referring to FIG. 7.

First, the surface of the surgical prosthesis is dipped in a mixture solution of APTES, which is an aminosilane compound, and ethanol (S320). Then, excess APTES is removed from the surface of the prosthesis dipped in the mixture solution of APTES and ethanol using an ultrasonic homogenizer (S340), and the surface of the prosthesis is annealed under a high-temperature environment (S360).

Referring again to FIG. 6, a second coating layer is formed on the surface of the surgical prosthesis coated with the first coating layer 110 having amino groups by the first or second method (S400). In an exemplary embodiment, the second coating layer 120 may be coated on the first coating layer 110 by dipping the surface of the prosthesis coated with the first coating layer in a mixture solution of at least one fluorocarbon containing carboxyl groups such as perfluoroalkane, perfluorodialkyl ether and perfluorotrialkylamine.

Next, a third coating layer is formed on the surface of the surgical prosthesis with the second coating layer 120 coated (S500). In an exemplary embodiment, the third coating layer 130 may be coated on the second coating layer 120 by dipping the surface of the prosthesis with the second coating layer coated in a liquid perfluorocarbon, which is a lubricant fluid.

### MODE FOR INVENTION

### Preparation Example 1

Step 1: An orthopedic prosthesis was immersed in an acetone solution and then washed for 15 minutes using an ultrasonic homogenizer. Then, the orthopedic prosthesis was immersed in an alcohol solution and then washed for 15 minutes using an ultrasonic homogenizer. The washed orthopedic prosthesis was taken out and the surface of the prosthesis was dried. The surface-dried prosthesis was immersed in a deionized water solution and washed for 15 minutes using an ultrasonic homogenizer.

Step 2: Micro/nano-sized surface roughness was formed on the surface of the orthopedic prosthesis washed in the step 1 by spraying polygonal crushed grits with a size of 2.5 m for 200 seconds.

Step 3: A first coating layer was coated by dipping the surface of the orthopedic prosthesis with the surface roughness formed in the step 2 in a mixture solution of 2 mg/mL dopamine hydrochloride, 1.347 mg/mL copper sulfate (CuSO₄.5H₂O), 2.2 μL/mL hydrogen peroxide and 50 mM Tris buffer for 20 minutes at room temperature.

Step 4: A second coating layer was coated by curing the orthopedic prosthesis with the first coating layer formed in the step 3 in a mixture solution of 9% perfluorodecanoic acid and a 91% perfluoroalkane solvent for 1 hour under a condition of 80° C. or higher.

Step 5: A third coating layer was coated by dipping the orthopedic prosthesis with the second coating layer coated in the step 4 in a liquid perfluorocarbon for 10 minutes at room temperature.

### Preparation Example 2

Step 1: An orthopedic prosthesis was immersed in an acetone solution and then washed for 15 minutes using an ultrasonic homogenizer. Then, the orthopedic prosthesis was immersed in an alcohol solution and then washed for 15 minutes using an ultrasonic homogenizer. The washed orthopedic prosthesis was taken out and the surface of the prosthesis was dried. The surface-dried prosthesis was immersed in a deionized water solution and washed for 15 minutes using an ultrasonic homogenizer.

Step 2: Micro/nano-sized surface roughness was formed on the surface of the orthopedic prosthesis washed in the step 1 by spraying polygonal crushed grits with a size of 2.5 μm for 200 seconds.

Step 3: Hydroxyl (—OH) functional groups were formed on the surface of the prosthesis by irradiating oxygen plasma onto the surface of the orthopedic prosthesis with the micro/nano-sized surface roughness formed. Then, the surface of the orthopedic prosthesis with the surface roughness formed in the step 2 was dipped in a mixture solution of 5% APTES (3-aminopropyltriethoxysilane) and 95% ethanol for 60 minutes at room temperature. Subsequently, APTES not bound to the hydroxyl groups on the surface of the orthopedic prosthesis was removed using an ultrasonic homogenizer, and the orthopedic prosthesis was annealed under a condition of 60° C. or higher.

Step 4: A second coating layer was coated by curing the orthopedic prosthesis with the first coating layer formed in the step 3 in a mixture solution of 9% perfluorodecanoic acid and a 91% perfluoroalkane solvent for 1 hour under a condition of 80° C. or higher.

Step 5: A third coating layer was coated by dipping the orthopedic prosthesis with the second coating layer coated in the step 4 in a liquid perfluorocarbon for 10 minutes at room temperature.

### Test Example 1

After placing the surface-modified orthopedic prosthesis prepared as described above in a medium, methicillin-resistant Staphylococcus aureus was cultured under a condition of 37° C. for 72 hours. FIG. 8 shows a result of incubating the coated surface of the orthopedic prosthesis and observing the surface of the orthopedic prosthesis by fluorescence microscopy after fixation and staining.

### Test Example 2

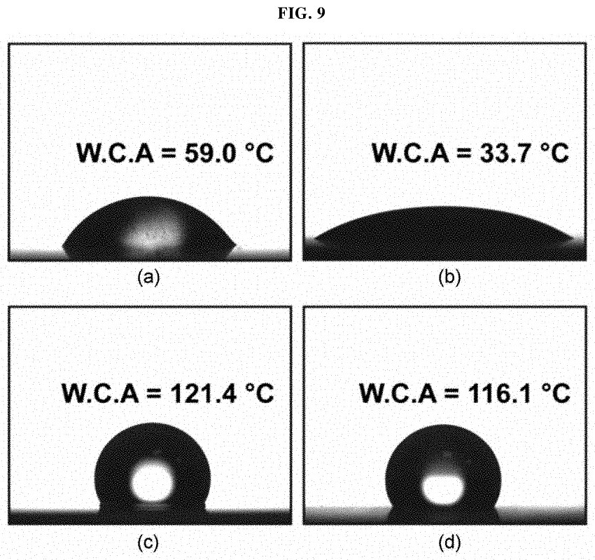

FIG. 9 shows a result of dropping about 5 μL of a liquid on the surface of the orthopedic prosthesis and measuring the contact angle on the surface of the prosthesis while tilting the surface. In FIG. 9, (a) shows the state of the surface before coating, (b) shows the state of the surface after the coating of the first coating layer, (c) shows the state of the surface after the coating of the second coating layer on the first coating layer, and (d) shows the state of the surface after the coating of the third coating layer on the second coating layer.

The surface-modified surgical prosthesis of the present disclosure described above can be used for treatment of bone fracture (as a metal nail or a plate for fixing bone). For example, when the prosthesis is inserted into the bone marrow or fracture site to fix the fracture site, acute infection that may occur due to contamination can be prevented fundamentally and effectively. It takes 6-12 months until the fracture heals completely. During the treatment period, chronic infection may occur if the immunity of the patient is lowered and the bacteria existing in the body are attached to the prosthesis. When the surface-modified prosthesis of the present disclosure is used, the risk of chronic infection may also be prevented because the attachment of biomaterials onto the surface of the prosthesis can be prevented for a long period of time.

The surface-modified surgical prosthesis of the present disclosure may also be used to treat a worn joint (artificial joint). Joint replacement is a surgery for replacing the joint damaged due to abrasion with an artificial joint made of metal, plastic, ceramic, etc. to maintain its function. However, the artificial joint requires revision surgery because of short lifetime due to abrasion. The surface-modified surgical prosthesis of the present disclosure can provide extended lifetime because abrasion is minimized by the third coating layer, and can reduce the pain of a patient by preventing the attachment of inflammatory factors and thereby minimizing inflammatory responses. In addition, the joint is vulnerable to infection because load is concentrated and inflammatory responses occur actively. When the surface-modified prosthesis of the present disclosure is used for the artificial joint, chronic infection by bacteria floating in the body can be prevented.

The foregoing description has been provided only to illustrate the present disclosure, and those having ordinary knowledge in the art to which the present disclosure belongs will be able to make various changes, modifications and substitutions without departing from the scope of the present disclosure. Accordingly, the examples disclosed in the present disclosure and the attached drawings are not for limiting but for describing the present disclosure, and the scope of the present disclosure is not limited by the examples and the attached drawings. The scope of the present disclosure should be interpreted based on the appended claims and it should be understood that all the equivalents within the scope are included in the scope of the present disclosure.

### Detailed Description of Main Elements

10: orthopedic prosthesis

110: first coating layer

120: second coating layer

130: third coating layer

### INDUSTRIAL APPLICABILITY

The present disclosure relates to a surface coating structure of a surgical prosthesis, surface-modified surgical prosthesis and a method for modifying the surface of a surgical prosthesis based thereon. It is expected that the surgical prosthesis will be used variously for preparation of prostheses in the medical devices market because the bacterial infection of prostheses can be prevented.

1. A surface coating structure of a surgical prosthesis, comprising:
a first coating layer formed on the surface of the surgical prosthesis and comprising an amino compound for surface adhesion;
a second coating layer formed on one side of the first coating layer and comprising a fluorine compound conferring hydrophobicity to the surface coating structure of the surgical prosthesis; and
a third coating layer formed on one side of the second coating layer and comprising a lubricant component for preventing adhesion of a biomaterial existing in a subject into which the surgical prosthesis is inserted.

2. The surface coating structure of a surgical prosthesis of claim 1, wherein the amino compound comprises a polydopamine or aminosilane compound.

3. The surface coating structure of a surgical prosthesis of claim 2,
wherein the polydopamine is dopamine hydrochloride,
wherein the first coating layer is formed by applying a mixture solution of the dopamine hydrochloride, copper sulfate and hydrogen peroxide to the surgical prosthesis.

4. The surface coating structure of a surgical prosthesis of claim 2,
wherein the aminosilane compound is 3-aminopropyltrimethoxysilane (APTES),
wherein the first coating layer is formed by applying a mixture solution of the 3-aminopropyltrimethoxysilane and ethanol to the surgical prosthesis and further comprises hydroxyl groups formed through an oxygen plasma process.

5. The surface coating structure of a surgical prosthesis of claim 1,
wherein the fluorine compound is a fluorocarbon compound,
wherein the second coating layer is formed by applying a mixture solution of the fluorocarbon compound bound to a carboxylic acid to the surgical prosthesis coated with the first coating layer.

6. The surface coating structure of a surgical prosthesis of claim 1, wherein the lubricant component comprises a substance selected from the group consisting of perfluorotri-n-pentylamine, perfluoropolyether, perfluorodecalin, perfluorohexane, perfluorooctane, perfluorooctyl bromide, perfluoroperhydrophenanthrene, and perfluorodecalin.

7. The surface coating structure of a surgical prosthesis of claim 1, wherein the first coating layer is formed to have a thickness of 30-50 nanometers (nm).

8. The surface coating structure of a surgical prosthesis of claim 1, wherein the first coating layer is coated on the surface of the surgical prosthesis with surface roughness formed by spraying polygonal crushed grits onto the surface of the surgical prosthesis together with compressed air.

9. A surface-modified prosthesis comprising:
a prosthesis inserted into a fracture site to fix the fracture site;
a first coating layer formed on the surface of the prosthesis and comprising an amino compound for surface adhesion;
a second coating layer formed on one side of the first coating layer and comprising a fluorine compound conferring hydrophobicity to the surface coating structure of the surgical prosthesis; and
a third coating layer formed on one side of the second coating layer and comprising a lubricant component for preventing adhesion of a biomaterial existing in a subject into which the surgical prosthesis is inserted.

10. The surface-modified prosthesis of claim 9,
wherein the amino compound is dopamine hydrochloride,
wherein the first coating layer is formed by applying mixture solution of the dopamine hydrochloride, copper sulfate, hydrogen peroxide and a Tris buffer to the surgical prosthesis.

11. A method for modifying the surface of a surgical prosthesis, comprising:
a step of forming a first coating layer comprising an amino compound for surface adhesion on the surface of a surgical prosthesis for producing a surface coating structure of the surgical prosthesis;
a step of forming a second coating layer comprising a fluorine compound conferring hydrophobicity to the surface coating structure of the surgical prosthesis on one side of the first coating layer; and
a step of forming a third coating layer comprising a lubricant component for preventing adhesion of a biomaterial existing in a subject into which the surgical prosthesis is inserted on one side of the second coating layer.

12. The method for modifying the surface of a surgical prosthesis of claim 11,
wherein the amino compound is dopamine hydrochloride,
wherein the first coating layer is formed by applying a mixture solution of the dopamine hydrochloride, copper sulfate, hydrogen peroxide and a Tris buffer to the surgical prosthesis.

13. The method for modifying the surface of a surgical prosthesis of claim 11, further comprises:
a step of pretreating the surface of the surgical prosthesis with at least one of acetone, alcohol and deionized water to remove organic or inorganic materials present on the surface of the surgical prosthesis; and
a step of forming surface roughness on the pretreated surface of the surgical prosthesis by spraying polygonal crushed grits onto the pretreated surface of the surgical prosthesis together with compressed air,
wherein the first coating layer is formed on the surface of the prosthesis with the surface roughness formed.

14. The method for modifying the surface of a surgical prosthesis of claim 11, wherein the lubricant component comprises a substance selected from the group consisting of perfluorotri-n-pentylamine, perfluoropolyether, perfluorodecalin, perfluorohexane, perfluorooctane, perfluorooctyl bromide, perfluoroperhydrophenanthrene, and perfluorodecalin.

15. The method for modifying the surface of a surgical prosthesis of claim 11, wherein the first coating layer is formed to have a thickness of 30-50 nanometers (nm).
